(19) United States
(12) Patent Application Publication
(10) Pub. No.: US 2005/0192296 A1
(43) Pub. Date: Sep. 1, 2005

(54) Process for the preparation of valacyclovir hydrochloride

(76) Inventors: Zvi Harel, Kfar Saba (IL); Michael Pesachovich, Givat-Shmuel (IL)

Correspondence Address: KENYON & KENYON, ONE BROADWAY, NEW YORK, NY, 10004, US

(21) Appl. No.: 11/040,925

(22) Filed: Jan. 21, 2005

Related U.S. Application Data
(60) Provisional application No. 60/538,362, filed on Jan. 21, 2004.
(60) Provisional application No. 60/591,707, filed on Jul. 27, 2004.

(51) Int. Cl. C07D 473/10; A61K 31/522

(52) U.S. Cl. 514/263.38; 544/276

(57) ABSTRACT

Provided are HPLC methods for analyzing BOC-L-alanine in BOC-L-valine and alanine analogues in valacyclovir hydrochloride and a use and method of selecting valacyclovir compositions.

### CROSS-REFERENCE TO RELATED APPLICATIONS

This application claims the benefit of U.S. Provisional Application Nos. 60/538,362 filed on Jan. 21, 2004 and 60/591,707 filed on Jul. 27, 2004, the disclosures of which are incorporated by reference in their entirety herein.

### FIELD OF THE INVENTION

This invention relates to a synthesis of valacyclovir hydrochloride composition containing a low concentration of alanine analogues from the starting material BOC-L-valine containing a low concentration of BOC-alanine as determined by liquid-solid chromatography.

### BACKGROUND OF THE INVENTION

Valacyclovir (Formula I) is an L-valyl ester prodrug of acyclovir (Formula II), an acyclic analog of a natural nucleoside. Acyclovir is reported to have high anti-viral activity, and is widely used in the treatment and prophylaxis of viral infections in humans, especially infections caused by herpes viruses. See Goodman and Gilman's, THE PHARMACOLOGICAL BASIS OF THERAPEUTICS 1193-1198 (9th ed. 1996).

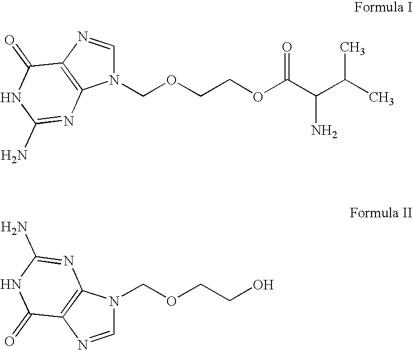

Processes for synthesizing valacyclovir hydrochloride can employ valine having an amine-protecting group, such as a t-butoxycarbonyl group (t-BOC). For example, U.S. Patent Application 20030153757 discloses a method of synthesizing valacyclovir hydrochloride using an amine protected valine as a starting material. BOC-L-valine, useful as a starting material for synthesis of valacyclovir, can contain impurities such as BOC-alanine. Such impurities in the starting material are undesirable because a final synthetic product obtained from such a starting material can be contaminated by alanine analogues of valacyclovir.

Impurities can be detected and quantified by HPLC.

### SUMMARY OF THE INVENTION

In one aspect, the present invention provides a method of synthesizing a valacyclovir hydrochloride composition comprising less than about 0.2 area-% alanine analogues, by employing as starting material BOC-L-valine having less than about 0.2 area-% BOC-L-alanine. Preferably, the starting material employed contains less than about 0.1 area-% BOC-L-alanine, and the valacyclovir hydrochloride composition contains less than about 0.1% area-% alanine. Most preferably, the starting material employed contains less than about 0.05 area % area-% BOC-L-alanine, and the valacyclovir hydrochloride composition contains a non-detectable amount of alanine derivative.

In another aspect, the present invention also provides a liquid-solid chromatographic method for determining the concentration of BOC-alanine in BOC-L-valine, the concentration of alanine analogues in crude valacyclovir hydrochloride and the final product, as well as crystalline valacyclovir hydrochloride.

### DETAILED DESCRIPTION OF THE INVENTION

As used herein, “gradient elution” refers to the change in the composition of the gradient eluent over a fixed period of time, stepwise or at a constant rate of change, as the percentage of the first eluent is decreased while the percentage of the second eluent is increased.

As used herein, “gradient eluent” refers to an eluent composed of varying concentrations of first and second eluents.

As used herein, “sample” refers to a small quantity or aliquot removed from a larger quantity, or batch, of either BOC-L-valine or valacyclovir hydrochloride, that is analyzed to estimate the characteristics of the larger quantity, or batch.

As used herein, in connection with a quantity of BOC-L-valine or valacyclovir hydrochloride, the term “batch” refers to a quantity from which a sample is taken. A batch is a mass obtained from a unit process or unit operation. The order of magnitude of the mass will depend on, among other things, the equipment used.

As used herein, the term “solid oral dosage forms” refers to capsules and tablets.

As used herein, the term “dry blend” refers to a mixture of valacyclovir hydrochloride and at least one excipient.

As used herein, the term, “detectable” refers to a measurable quantity measured using an HPLC method having a detection limit of 0.01 area-%.

As used herein, in connection with amounts of alanine analogues in valacyclovir hydrochloride, the term “not detectable” means not detected by the herein described HPLC method having a detection limit for alanine analogues of 0.01 area-%.

As used herein, the term “alanine analogues” includes valacyclovir-like molecules in which the moiety attached to the hydroxyethoxyrnethyl group is alanine and not valine.

As used herein, in connection with a measured quantity, the term “about” refers to that variation in the measured quantity as would be expected by the skilled artisan making the measurement and exercising a level of care commensurate with the objective of the measurement and the precision of the measuring equipment used.

As used herein, the term “area-%” refers to a comparison of the area under the peak (hereinafter “AUP”) for each analyte as measured by the detector, for example on a chromatogram, during the liquid-solid chromatographic analysis. AUP can be determined by using a suitable integrator. Each peak in the chromatogram corresponds to a different component in the mixture which was loaded onto the liquid-solid chromatographic column, and the ratio of the AUP of each of the detectable components with the total AUPs of all the sample components results in the area percentage. Area percent can be expressed mathematically as:
areaᵢ-%=100×(AUPᵢ)/(Σ all AUPs)

Valacyclovir hydrochloride compositions consist essentially of valacyclovir hydrochloride.

Valacyclovir hydrochloride can be prepared using BOC-L-valine as a starting material, by methods such as described in U.S. publication no. 2003/0153757, hereby incorporated by reference. BOC-L-valine can be contaminated with BOC-alanine. The amount of alanine analogues present in the intermediate crude product and crystalline end product can be manipulated by, among other things, using a starting material having low levels of BOC-alanine, especially a low level of BOC-L-alanine. The levels of BOC-alanine can be determined by liquid-solid chromatography.

Liquid-solid chromatography, especially high pressure liquid chromatography, also known as high performance liquid chromatography, (hereinafter “HPLC”) has been applied to the detection and quantification of impurities in a chemical compound. In HPLC, the components to be separated and measured, commonly known or referred to as analytes, are dissolved in a diluent (solvent) that can be the same as the eluent, or the mobile liquid phase through the column. The mobile liquid phase and dissolved analytes interact with a packing in the column commonly denoted the stationary phase. Because the different analytes interact differently with the stationary phase, each analyte will transverse the column at a different rate. See 13 JAMES D. WINEFORDNER, TREATISE ON ANALYTICAL CHEMISTRY, pt. I (2d ed. 1993).

Reversed phase HPLC utilizes a nonpolar stationary phase and a polar eluent. Gradient elution improves separation of sample components by changing the composition of the mobile phase, or gradient eluent, over time. A detector is used to monitor the separation by measuring a particular physical property of the eluent. For example, a spectrophotometer can be used as a detector by measuring the radiation absorbance of the mobile phase.

Applicants have discovered that the concentration of alanine analogues in the final valacyclovir or valacyclovir hydrochloride product can be manipulated by, among other things, controlling the concentration of BOC-alanine in the starting material, BOC-L-valine. There is a need for methods of detecting alanine analogues in the starting materials, intermediates, and final products of valacyclovir hydrochloride synthesis. There is also a need for valacyclovir hydrochloride containing a low concentration of alanine analogues.

In one embodiment, the present invention provides a liquid-solid chromatographic method for determining the concentration of BOC-alanine in BOC-L-valine. The concentration of the BOC-alanine contaminant in BOC-L-valine can be measured by liquid-solid chromatography, preferably through HPLC, and in particular by the herein below described HPLC method. HPLC uses a suitable chromatography column, such as the reverse phase column Inertsil ODS-3V 5 μm 150×4.6 mm (GL Sciences, Cat. No. 5020-01731).

The first step of the HPLC method of the present invention for measuring the amount of BOC-L-alanine in BOC-L-valine includes loading a sample of BOC-L-valine onto a liquid-solid chromatography column. Loading can be effected by injecting a solution of the sample onto the column. A suitable volume of material for injection onto the column is about 50 μL. The diluent used to make the solution of the sample for injection can be, for example, the eluent. The column can be at ambient temperature, preferably at about 25° C. The column stationary phase can be modified silica gel preferably 5 μm, Spherical silica gel bonded with octadecyl groups, endcapped with 15% Carbon loading, and is preferably Inertsil ODS-3V. After the sample is loaded onto the column, the column is then isocratically eluted with eluent. The preferred eluent is a solution of acetonitrile (27%) and water containing 0.05% phosphoric acid (0.5 g, 85% H₃PO₄/1L H₂O) (73%), at a constant flow rate of no greater than about 1 mL/min. The response of a UV detector to the column effluent is monitored, wherein the UV detector can be a spectrophotometer operating in the range of 200-600 nm, preferably at 210 nm. On the basis of the detector response to the eluted components, the amount of BOC-alanine in BOC-L-valine is calculated as area-%.

The suitability of the HPLC system can be checked with a system suitability solution that includes a mixture of BOC-alanine (0.15 mg/mL) and BOC-L-valine (15 mg/mL) in diluent.

In another embodiment, the present invention provides a liquid-solid chromatographic method for determining the amount of alanine analogues present in a sample of valacyclovir hydrochloride. Through liquid-solid chromatography, especially through HPLC, the concentration of alanine analogues can be measured. A suitable chromatography column for such measurement is the reverse phase column Inertsil ODS-3V 5 μm, or an equivalent. Preferably, the method employs gradient elution. This process allows for more effective separation of sample components.

The first step of the HPLC method of the present invention for measuring the amount of alanine analogues in valacyclovir hydrochloride includes loading the sample of valacyclovir hydrochloride onto a liquid-solid chromatography column. Loading can be effected by injecting a solution of the sample onto the column. When loading is by injection, the injection volume is about 20 μL. Additionally, the diluent used to make the solution of the sample for injection can be, for example, the same as the first eluent. The column temperature can be greater than room temperature. Preferably the column temperature is about 30° C. The column stationary phase can be modified silica gel preferably 5 μm, Spherical silica gel bonded with octadecyl groups, endcapped with 15% Carbon loading, and is preferably Inertsil ODS-3V 1. The column is then gradient eluted at a gradient eluent flow rate no greater than about 1.5 mL/min, with a gradient eluent having first and second eluents.

A suitable first eluent is a 0.01M solution of potassium dihydrogen phosphate in water (98%) and acetonitrile (2%). The pH of the first eluent is acidic, preferably having a pH value of about 3.5. The pH can be adjusted using 10% phosphoric acid. A suitable second eluent is acetonitrile. An equilibration time of about 7 minutes is usually suitable. The response of a UV detector to the column effluent is monitored, wherein the UV detector can be a spectrophotometer operating in the range of 200-600 nm, preferably at 254 nm. On the basis of detector response, the amount of alanine analogues in valacyclovir hydrochloride is calculated as area-%.

A system suitability solution can be prepared by dissolving valacyclovir in a guanine solution and an acyclovir solution. The sample solution can be a concentration of 0.8 mg/mL valacyclovir in diluent. The sample solution can be injected onto the column, and the concentration of any impurity can then be measured using a suitable integrator to determine the area-% of each mixture component.

In another embodiment, the present invention provides a method for synthesizing a valacyclovir hydrochloride composition that comprises an amount of alanine analogues of less than about 0.2 area-% but greater than or equal to about 0.01 area-%, which method includes the steps of:
a) obtaining one or more samples of one or more BOC-L-valine batches;
b) measuring the level of BOC-L-alanine in each of the samples of step (a);
c) selecting the BOC-L-valine batch or batches that comprise a less than about 0.2 area-% of BOC-L-alanine based on the measurement or measurements conducted in (b); and
d) using the batch selected in (c) to synthesize said valacyclovir hydrochloride composition.

Preferably, the BOC-L-valine sample and the valacyclovir hydrochloride obtained contain, respectively, BOC-L-alanine and alanine analogue in an amount of less than about 0.1 area-%. Most preferably, when the BOC-L-valine sample contains less than 0.05 area-% BOC-L-alanine, the valacyclovir hydrochloride composition contains a non-detectable amount of alanine.

The level of BOC-L-alanine in the BOC-L-valine sample is determined using the liquid-solid chromatographic methods described above or by equivalent methods.

Specifically, the present invention provides a method for synthesizing a valacyclovir hydrochloride composition that comprises less than about 0.2 area-% alanine analogue. The first step of this synthetic method includes analyzing at least one sample of BOC-L-valine of one or more BOC-L-valine batches for presence of its alanine analogue as an impurity, and selecting a batch that contains less than about 0.2 area-% alanine analogue. The selected BOC-L-valine is reacted with acyclovir in an organic solvent, preferably a solution of dicyclohexylcarbodiimide (hereinafter “DCC”) in dimethylformamide (hereinafter “DMF”) to obtain a mixture. The mixture is then combined with 4-dimethylaminopyridine (hereinafter “DMAP”), and then water to obtain a suspension. The precipitate, dicyclohexyl urea, is removed by filtration and the resulting filtrate is then concentrated. The filtrate is then reconstituted in, or dissolved in a lower alcohol at reflux, especially isopropyl alcohol, to obtain protected valacyclovir. This can then be deprotected and recrystallized from water and isopropyl alcohol to provide crystalline valacyclovir hydrochloride. The synthesis of valacyclovir hydrochloride can be achieved on different scales, provided the weight volume ratio is maintained for all reactants.

The valacyclovir hydrochloride obtained by the present invention may be formulated into pharmaceutical compositions. In addition to the active ingredient(s), the pharmaceutical formulations of the present invention can and typically do contain one or more excipients. The formulations are typically prepared in a batchwise manner and are processed into solid oral dosage forms, for example tablets and capsules. Release of solid oral dosage forms in to the stream of commerce can be based on, among other things, the level of alanine analogues in the valacyclovir hydrochloride, in the dry blend, or in the solid oral dosage forms.

Excipients are added to the formulation for a variety of purposes. Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc, to mention just a few.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

For quality control purposes, it is preferred that the pharmaceutical compositions be made from valacyclovir hydrochloride that has a low level of alanine. Typically pharmaceutical compositions are made in batches or lots for production purposes. A production lot should be checked to insure that the level of the alanine analogue is within specification; i.e., a quality control test. A sample from the production lot (e.g. 10 to 100 capsules or tablets) is taken and assayed for the presence of the alanine analogue and preferably also for the content of the same. Generally the entire production lot, minus any retained sample(s), will be sold or otherwise released by the manufacturer unless an unacceptable level of the alanine analogue is found. In that case, the production lot will not be sold or released; i.e. neither placed in commerce nor used in clinical studies. The same strategy can be applied for production lots of valacyclovir hydrochloride substance.

The present invention in certain of its embodiments will now be illustrated by the following non-limiting examples.

### EXAMPLE 1

This example demonstrates a liquid-solid chromatographic method for determining the concentration of BOC-alanine in BOC-L-valine.

A solution to test system suitability was prepared using 0.15 mg/mL of BOC-alanine and 15 mg/mL of BOC-L-valine in diluent. The diluent used was the same as the eluent, comprised of 73% of 0.05% of phosphoric acid in water, and 27% acetonitrile. A sample volume of 50 μL was loaded onto an Inertsil ODS-3V 5 μm 150×4.6 mm column at 25° C. The detector was set at 210 nm and the sample was eluted at a flow rate of 1 mL/min. Retention times for BOC-alanine and BOC-L-valine were 6 minutes and 14.5 minutes, respectively. AUPs were then compared using a suitable integrator to confirm area-% concentration of BOC-alanine in BOC-L-valine.

### EXAMPLE 2

This example demonstrates a liquid-solid chromatographic method for determining the concentration of alanine analogues in valacyclovir hydrochloride.

A solution to test system suitability was prepared by dissolving guanine (5 mg) in 0.2N NaOH (10 mL). The solution was then diluted further to 100 mL with 98% 0.01M potassium dihydrogen phosphate in water adjusted to pH=3.5 with 10% phosphoric acid and 2% acetonitrile. A second solution of acyclovir (5 mg) in diluent to a total volume of 100 mL was also prepared. 2 mLs of each solution were then added to valacyclovir hydrochloride (20 mg). The total volume of the valacyclovir hydrochloride solution was brought up to 25 mL with diluent. This diluent also served as the first eluent for HPLC evaluation. The second eluent was acetonitrile. 20 uL of the valacyclovir hydrochloride, guanine, acyclovir mixture was injected onto an Inertsil ODS-3V 5 μm 250×4.6 mm column. The column was eluted on a gradient of 0-20% second eluent; 32 min. at a flow rate of 1.5 mL/min at a temperature of 30° C. The detector was set at 254 nm. The retention time of valacyclovir hydrochloride was 13 min. The resolution between the guanine and the acyclovir should not be less than 15.0, and a tailing factor of not more than 4.0 for valacyclovir hydrochloride should be achieved.

### EXAMPLE 3a

This example describes the formation of protected valacyclovir in a synthesis to produce valacyclovir hydrochloride having less than about 1.4 area-% alanine analogues.

BOC-L-valine (870 g) having less than about 1 area-% BOC-alanine was fully dissolved in DMF (5874 mL) under nitrogen, with stirring. The mixture was then cooled to −5° C. A solution of DCC (330 g) in DMF (600 g) was added to the mixture during 20 min, and the obtained mixture was stirred at −5° C., 20 min. Acyclovir (600 g) was added to the mixture, and after 5 min of stirring, DMAP (98 g) was added. The mixture was stirred at −5° C., 3 h. DCC (330 g) in DMF (600 g) was added during 20 min, and the obtained mixture was stirred at −5° C., 3 h. DCC (438 g) in DMF (780 g) was added during 20 min, and the obtained mixture was stirred at −5° C., 3 h. The mixture was heated to 25° C. during 2.5 h, and stirred 4 h. Water (204 g) was added, and the mixture was stirred at 25° C., 4 h. The resulting precipitate, dicyclohexyl urea, was recovered by filtration and washed with DMF (1800 g). The filtrate was then concentrated under reduced pressure (10 mmHg) to obtain a residue. This residue reconstituted by dissolution at reflux in isopropyl alcohol (hereinafter “IPA”) (6120 g). The mixture was cooled to 25° C., and the resulting precipitate, protected valacyclovir was recovered by filtration.

### EXAMPLE 3b

This example describes the deprotection of protected valacyclovir hydrochloride in a synthesis to produce valacyclovir hydrochloride having less than about 1.4 area-% alanine analogues.

Protected valacyclovir (578 g, on a dry basis) was dissolved in formic acid (1440 mL) at 25° C. Water (186 mL) was added to the mixture, and then a solution of 32% HCl (311 g) was added during 1 h. The mixture was stirred at 25° C., 1-5 h until the concentration of protected valacyclovir was reduced to 0.5% or less. IPA (9200 mL) was added to the mixture during 30 min, and the mixture was cooled to −5° C. The resulting precipitate was recovered by filtration, yielding crude valacyclovir hydrochloride having less than about 1.4 weight-% alanine analogues.

### EXAMPLE 3c

This example describes the formation of crystalline valacyclovir hydrochloride having less than about 1.4 area-% alanine analogues.

Crude valacyclovir hydrochloride (380 g) was dissolved in water (1520 mL) at 40° C. The mixture was filtered and cooled to 35° C. IPA (5700 mL) was added to the mixture during 3 h. The mixture was cooled to −5° C. The resulting precipitate, crystalline valacyclovir hydrochloride, was recovered by filtration. The wet precipitate was dried under vacuum, and the dry precipitate was milled. The crystalline valacyclovir hydrochloride having less than about 1.4 area-% alanine analogues.

### EXAMPLE 4a

This example describes the formation of protected valacyclovir in a synthesis to produce valacyclovir hydrochloride having less than about 0.03 area-% alanine analogues.

BOC-L-valine (870 g) having less than about 0.05% BOC-alanine was dissolved in DMF (5874 mL) under nitrogen, and stirred at 20-25° C. until fully dissolved. The mixture was then cooled to −5° C. A solution of DCC (330 g) in DMF (600 g) was added to the mixture during 20 min, and the obtained mixture was stirred at −5° C., 20 min. Acyclovir (600 g) was added to the mixture, and after 5 min of stirring, DMAP (98 g) was added. The mixture was stirred at −5° C., 3 h. DCC (330 g) in DMF (600 g) was added during 20 min, and the obtained mixture was stirred at −5° C., 3 h. DCC (438 g) in DMF (780 g) was added during 20 min, and the obtained mixture was stirred at −5° C., 3 h. The mixture was heated to 25° C. during 2.5 h, and stirred 4 h. Water (204 g) was added, and the mixture was stirred at 25° C. for 4 hours. The resulting precipitate, dicyclohexyl urea, was recovered by filtration and washed with DMF (1800 g). The filtrate was then concentrated under reduced pressure (10 mmHg) to obtain a residue. This residue was dissolved at reflux in IPA (6120 g). The mixture was cooled to 25° C., and the resulting precipitate, protected valacyclovir was recovered by filtration.

### EXAMPLE 4b

This example describes the formation of crude valacyclovir hydrochloride in a synthesis to produce valacyclovir hydrochloride having less than about 0.03 area-% alanine analogues.

The protected valacyclovir obtained from the process described in Example 4a. (578 g, on a dry basis) was dissolved in formic acid (1440 mL) at 25° C. Water (186 mL) was added to the mixture, and then a solution of 32% HCl (311 g) was added during 1 h. The mixture was stirred at 25° C., 1-5 h, until the concentration of protected valacyclovir was reduced to 0.5% or less. IPA (9200 mL) was added to the mixture during 30 min, and the mixture was cooled to −5° C. The resulting precipitate was recovered by filtration, yielding crude valacyclovir hydrochloride having less than about 0.03 area-% alanine analogues.

### EXAMPLE 4c

This example describes the formation of crystalline valacyclovir hydrochloride having less than about 0.03 area-% alanine analogues.

Crude valacyclovir hydrochloride (380 g) was dissolved in water (1520 mL) at 40° C. The mixture was filtered and cooled to 35° C. IPA (5700 mL) was added to the mixture during 3 h. The mixture was cooled to −5° C. The resulting precipitate, crystalline valacyclovir hydrochloride, was recovered by filtration. The wet precipitate was dried under vacuum, and the dry precipitate was milled, yielding crystalline valacyclovir hydrochloride having no detectable alanine analogues.

1. A process for preparing a valacyclovir hydrochloride composition containing less than about 0.2 area-% alanine analogues comprising:
a) obtaining one or more samples of one or more batches of BOC-L-valine;
b) measuring the level of BOC-L-alanine in each of the samples of step (a);
c) selecting the BOC-L-valine batch having a level of BOC-L-alanine of less than about 0.2 area-% based on the measurement or measurements conducted in (b); and
d) using the batch selected in step (c) to synthesize the valacyclovir hydrochloride composition.

2. A process for preparing a valacyclovir hydrochloride composition containing less than about 0.2 area-% alanine analogues comprising:
a) measuring the BOC-L-alanine in a sample of BOC-L-valine, wherein the sample is selected from one or more batches of BOC-L-valine;
b) selecting a batch that contains less than about 0.2 area-% BOC-L-alanine;
c) reacting the selected BOC-L-valine with acyclovir in an organic solvent to obtain a mixture;
d) combining the mixture of step c) with 4-dimethylaminopyridine and then water to obtain a precipitate;
e) removing the precipitate of step d) and concentrating the resulting filtrate;
f) adding a lower alcohol to the concentrated filtrate of step e), at reflux to obtain protected valacyclovir;
g) deprotecting the protected valacyclovir of step f) in formic acid, water and HCl to obtain crude valacyclovir hydrochloride; and
h) recrystallizing the valacyclovir hydrochloride of step g) in water and isopropyl alcohol to obtain a composition having less than about 0.2 area-% alanine analogues.

3. The process of any one of claims 1 and 2, wherein the selected BOC-L-valine batch contains less than about 0.1 area-% BOC-L-alanine, and the valacyclovir hydrochloride composition obtained contains less than about 0.1 area-% of alanine analogues.

4. The process of claim 3, wherein the selected BOC-L-valine batch comprises about 0.05 area-% BOC-L-alanine, and the valacyclovir hydrochloride composition obtained comprises a non-detectable level of alanine analogues.

5. The process of claim 2, wherein the organic solvent in step (c) is a mixture of dicyclohexylcarbodiimide and dimethylformamide.

6. The process of claim 2, wherein the lower alcohol in step (f) is isopropyl alcohol.

7. The process of any one of claims 1 and 2, wherein the measuring of the BOC-L-valine sample is performed by a liquid-solid chromatographic process for determining the amount of BOC-L-alanine in a sample of BOC-L-valine, comprising the steps of:
a) loading the sample onto a liquid-solid chromatography column;
b) eluting the column with eluent at a constant flow rate of about 1 mL/min or less, wherein the eluent comprises about 27% acetonitrile and about 73% of 0.05% phosphoric acid in water;
c) monitoring the response of a UV detector to the column effluent wherein the UV detector operates in the range of 200-600 nm; and
d) calculating the amount, as area percentage, of BOC-L-alanine in BOC-L-valine on the basis of the detector response.

8. The process of claim 2, wherein the area-% of alanine derivatives in a sample of valacyclovir hydrochloride is measured by a liquid-solid chromatographic process, comprising the steps of:
a) loading the sample onto a liquid-solid chromatography column;
b) gradient eluting the column with a gradient eluent comprising first and second eluents, wherein the first eluent comprises 0.01M potassium dihydrogen phosphate in water (98%) and acetonitrile (2%), and the second eluent comprises acetonitrile, at a constant flow rate of about 1.5 mL/min;
c) monitoring the response of the UV detector to the column effluent wherein the UV detector operates in the range of 200-600 nm; and
d) calculating the amount, as area percentage, of the alanine analogues in valacyclovir hydrochloride on the basis of the detector response.

9. The process of any one of claims 7 and 8, wherein the column is a silica gel column.

10. The process of claim 8, wherein the pH of the first eluent is about 3.5.

11. The process of claim 8, wherein the column temperature is about 30° C.

12. A liquid-solid chromatographic process form measuring the amount of alanine analogues in valacyclovir hydrochloride, comprising the steps of:
a) loading the sample onto a liquid-solid chromatography column;
b) gradient eluting the column with a gradient eluent comprising first and second eluents, wherein the first eluent comprises 0.01M potassium dihydrogen phosphate in water (98%) and acetonitrile (2%), and the second eluent comprises acetonitrile, at a constant flow rate of about 1.5 mL/min;
c) monitoring the response of the UV detector to the column effluent wherein the UV detector operates in the range of 200-600 nm; and
d) calculating the amount, as area percentage, of the alanine analogues in valacyclovir hydrochloride on the basis of the detector response.

13. A quality-controlled distribution process for solid oral dosage forms of valacyclovir hydrochloride, comprising the steps of:
a) forming a dry blend batch of valacyclovir hydrochloride and at least one excipient,
b) processing the batch of step a) into a production lot of solid oral dosage forms of valacyclovir hydrochloride,
c) measuring the amount of alanine analogues in a sample from the production lot of step b) according to the process of claim 12, and
d) releasing the production lot of step b) into the stream of commerce if the amount of detectable alanine analogues measured in step c) is less than about 0.2 area-%.

14. A quality-controlled distribution process for solid oral dosage forms of valacyclovir hydrochloride, comprising the steps of:
a) measuring the amount of alanine analogues in a sample of valacyclovir hydrochloride according to the process of claim 12,
b) if the amount of detectable alanine analogues of step a) is less than about 0.2 area-%, forming a dry blend batch of the valacyclovir hydrochloride of step a) and at least one excipient,
c) processing the batch of step b) into a production lot of solid oral dosage forms of valacyclovir hydrochloride, and
d) releasing the production lot of step c) into the stream of commerce.

15. A process for preparing a pharmaceutical formulation of valacyclovir hydrochloride, comprising the steps of:
a) removing a sample from a batch of valacyclovir hydrochloride;
b) calculating the amount of alanine analogues in the sample by the process of claim 12; and
c) using the batch from which the sample contains less than about 0.2 area-% of detectable alanine analogues to prepare a pharmaceutical formulation of valacyclovir hydrochloride.

16. The process of claim 15 in which the batch and sample of step c) contain less than about 0.1 area-% alanine analogues.

17. The process of claim 15 in which the batch and sample of step c) contain less than about 0.05 area-% alanine analogues.

18. A process for preparing valacyclovir hydrochloride comprising the step of preparing valacyclovir hydrochloride starting with BOC-L-valine containing less than about 0.2 area-% detectable BOC-L-alanine.
